(19) United States
(12) Patent Application Publication
(10) Pub. No.: US 2025/0362261 A1
(43) Pub. Date: Nov. 27, 2025

(54) REVERSIBLE FUNCTIONALIZATION OF NANOPORES USING AN ADHESION LAYER

(71) Applicant: University of Rhode Island Board of Trustees, Kingston, RI (US)

(72) Inventors: Jason R Dwyer, Providence, RI (US); James Hagan, West Warwick, RI (US); Matthew Kiesewetter, North Kingston, RI (US); Kassie Picard, Kingston, RI (US); Brian Sheetz, North Kingston, RI (US)

(21) Appl. No.: 19/215,206

(22) Filed: May 21, 2025

Related U.S. Application Data
(60) Provisional application No. 63/651,340, filed on May 23, 2024.

(51) Int. Cl. G01N 27/12; C01B 21/068; G01N 33/487

(52) U.S. Cl. CPC G01N 27/128; C01B 21/0687; G01N 33/48721; C01P 2006/16

(57) ABSTRACT

A stable substrate is disclosed comprising one more nanopores coated with an adhesion layer of a stabilizing compound, covalently bound to the nanopore interior via at least one bonding site, and having at least one coupling site. The substrate further comprises a functional enhancement layer of coupling partner molecules bound to the adhesion layer with a bond between the stabilizing compound's coupling site and the coupling partner's coupling bonding site.

### CROSS-REFERENCE TO RELATED APPLICATION

This application claims the benefit of U.S. Provisional Patent Application No. 63/651,340, filed on May 23, 2024, which is incorporated by reference herein in its entirety.

### STATEMENT REGARDING FEDERALLY SPONSORED RESEARCH OR DEVELOPMENT

This invention was made with government support under grant number R21 HG011096 awarded by the National Institutes of Health and grant number 1808344 awarded by the National Science Foundation. The government has certain rights in the invention.

### BACKGROUND

Nanoscale devices offer the potential for atomic scale control of chemical reactions or active devices involving single molecules. One way this potential has been demonstrated is by creating nanopores with sizes so small that only one or a few molecules of interest can pass through the pore at one time and which provide highly selective interactions with chosen chemicals. These devices function through mechanical interactions or chemical transformations, or combination of both.

To realize this hypothetical potential, one needs to be able to reliably create, store, and make prolonged use of not only isolated nanopores, but also nanopore based systems. Engineering such nanopore systems even at experimental scale poses challenges related to stability, selectivity, sensing, and robustness. In some applications, creating only a few such pores could be adequate, though in other applications large numbers of such pores-perhaps on the order of a million pores-would have to be created to support sufficient throughput for full-scale commercial application such as bulk purification. Supporting such applications will require inexpensive and reproducible methods for mass production of useful and long-lived nanopores.

One approach to pore fabrication has been to create protein-based nano-pores disposed in lipid membranes. Lipid (bilayer) based pores and protein pores are likely to be delicate and subject to various mechanisms of degradation. These lipid bilayer membranes and protein pores are unlikely to be compatible with chemical systems that require organic, generally non-polar solvents typically dictated by modern synthetic chemistry. Protein-based pores are commercially available and while some are robust, these pores are of fixed size, can require complex preparations, can be hard to produce at large scale, and can be challenging to integrate with inorganic nanoscale devices. Hence, biological analog approaches are not ideal for real-world applications which require tuning effective pore sizes, would benefit from compatibility with efficient microfabrication methods, are scalable and robust, and which are directly integrable with other nanoscale devices.

In contrast, solid-state abiotic nanopores offer tunability, ruggedness,

manufacturability at scale, compatibility with a range of solvents, and are often created from abundant, commercially available inorganic substrates. Abiotic nanopores have been employed in, among other things, applications as high-performance platforms for single-molecule science, as nanoscale apertures for fundamental physics experiments and controlled cargo delivery, as conductive and often rectifying ionic circuit elements, as high-resolution model systems for nanoporous filters and even as potentially robust, device-ready analogues that mimic their proteinaceous brethren. Thus, development of abiotic nanopores provides a route with higher probability of success to a wide range of scientific and commercial applications.

As a general proposition, tailoring a nanopore for a specific application involves creating a pore with geometrical, ionic conductance, ion and molecular transport, physical, and chemical properties that will achieve a sufficiently specific response to a relevant class of analytes. For an example of geometric properties, it is possible to control overall pore size through initial fabrication techniques that create a pore in a substrate of a desired size. Additionally, the effective size of a pore can be controlled by binding a molecule of the right size and geometry to the pore interior to achieve a desired level of steric interference between any number of analytes and the pore. It is also possible to control how permeable a pore is by selecting molecules with desired levels of hydrophobicity to be bound to the pore interior. This can be done by binding a homogeneous layer of molecules having structures with an internally inhomogeneous charge distribution, or a heterogeneous layer of molecules with differing charge distributions (potentially including uniformly charged molecules) between them, to the pore interior such that the overall hydrophobicity of the pore is altered. Further control can be achieved by thoughtful selection of solvents or by the application of electrical gradients or electromagnetic fields. Other properties of bound molecules can be exploited, such as using molecules which can be photo-isomerized so that the molecules' three-dimensional configuration can be altered in situ by the external application of light. Yet another approach would be to use pre-selected molecules with very high binding specificity for a specific analyte bound to a nanopore surface to obtain precise control of sensing and switching. In such a case, the flow of current through a nanopore would change when a specific analyte binds with the pre-selected molecule. If the current change is big enough with binding/unbinding of an analyte, one can consider the introduction of the analyte to have switched the nanopore between two states. These methods offer the ability to tailor the effective pore size relative to different molecular analytes and tailoring the permeability of a pore as a function of analyte type—providing selectivity that could be used for, inter alia, filtering or creating logic circuits.

Tailored nanopore systems are typically purpose-built from scratch, starting with a thin substrate and using mechanical, electrical, or chemical means to fabricate raw nanopores in the substrate which is typically a membrane or film. Subsequently, chemical modifications are performed to further refine the desired functionality of the nanopore interiors. Creating nanopores with stability, consistent properties, and functional longevity is challenging. As already mentioned, a good starting point for robustness is starting with an (often silicon-rich) abiotic substrate. Fabrication conditions and substrate thickness are chosen to tailor the nanopore properties, and treatments may be applied after gross fabrication which further tailor nanopore properties. Constructing pores with the exact properties required is generally time-consuming and requires specialized techniques. In addition, achieving functionalization along with stability and robustness with custom-created nanopores has been challenging because each choice of functionalizing molecule interacts differently with the substrate, solvents, and analytes. It would be beneficial if an intermediate nanopore structure with predictable properties could provide a robust platform to predictably produce a diverse range of eventual functionalities.

Moreover, tailored nanopores are not typically designed to be reconfigurable such that a nanopore-based system could be retargeted to, for example, detect or selectively control the transport of a second and different analyte. These deficiencies limit the utility and ultimately slow down research.

For at least these reasons, it would be highly beneficial to exploit a system providing a common path to creating robust reconfigurable nanopore systems which are both initially stable and which provide flexible, low-overhead paths to further customization with diverse sets of chemical functions. Also desirable would be the use of a reasonably priced and readily available substrate such as silicon nitride.

Silicon nitride is readily available because of the existing electronics manufacturing supply chain; it is relatively safe to use, inexpensive, and well understood. Moreover, efficient fabrication of nanopores in silicon nitride is known in the art, and powerful techniques for functionalizing silicon nitride exist. Also, given the advanced technology base supporting silicon-based substrates, methods of creating devices in large numbers are likely to be achieved with silicon materials. For example, methods used in semiconductor processing are expected to be able to produce uniform arrays containing millions of nanopores.

Despite the numerous benefits of using silicon nitride substrates, it is well known that the susceptibility of silicon nitride to oxidation, hydrolysis, and dissolution creates serious problems. Silicon nitride hydrolyzes and forms an oxide that blocks the ability to conduct some preferred surface chemistry, and which can quickly degrade any pores created. Moreover, degradation products can contaminate an analytical system, effectively destroying system functionality.

Stabilizing silicon nitride can be done by using various approaches, according to various embodiments of the present invention. One approach is binding hydrophobic organic molecules to its surface through means such as photo-hydrosilylation. Another approach would be binding silane or silatrane to an oxidized silicon nitride surface.

Other substrates can also be used as the membrane to practice the invention. Compatible nanopores fully penetrating a membrane made from a range of “2D” materials (such as MXene, graphene, molybdenum disulfide, hexagonal boron nitride), polymers (e.g., those derived from petroleum and used to make a plastic), transition metal dichalcogenides, silicon, silicon oxide, and silicon nitride. Pores in all of these materials may also be employed with the same overall scheme described below for reconfigurable nanopores. Accordingly, different surface chemistry reactions would be employed for binding the stabilizing molecules to the selected substrate according to various embodiments of the present invention.

Conventional approaches employ a single pre-determined stabilizing molecule selected to provide targeted functionality. Some variability in function using organic molecules has been demonstrated by using organic molecules capable of photo-isomerization, but at present, a practitioner must have a full range of skills such as the handling of wafers or films, fabrication of pores, functionalization of pores, analytical cell fabrication, and other skills to successfully employ nanopores for numerous emerging applications.

It would be highly desirable if practitioners had access to reconfigurable, largely prefabricated, stable, well-characterized nanopore membranes that a non-specialist practitioner could exploit for a range of purposes.

The present disclosure adds such a reconfigurable nanopore scheme to the art.

Known in the art is a dielectric breakdown nanopore fabrication technique described by Dwyer, J. et al. (2019), Covalent Chemical Surface Modification of Surfaces with Available Silicon or Nitrogen, (see also, U.S. Pat. No. 10,519,035) both of which are incorporated herein by reference.

Also known in the art are methods to functionalize pores created through dielectric breakdown such as that described by Bandara et el. in ACS Materials and Interfaces (2019) 11, 30411-30420, “Chemically Functionalizing Controlled Dielectric Breakdown Silicon Nitride Nanopores by Direct Photohydrosilylation.”

Also known in the art is a method of bonding an organic molecule to a silicon nitride or silicon substrate through photo-hydrosilylation to fabricate photoswitchable binary nanopores which can provide selective electronic detection of single biomolecules. In “Photoswitchable Binary Nanopore Conductance and Selective Electronic Detection of Single Biomolecules under Wavelength and Voltage Polarity Control” (James T. Hagan, Alejandra Gonzalez, Yuran Shi, Grace G. D. Han, and Jason R. Dwyer, ACS Nano 2022 16 (4), 5537-5544, DOI: 10.1021/acsnano.1c10039), a nanopore formed on a silicon-rich silicon nitride surface or a silicon surface is fabricated through dielectric breakdown techniques, and then bonded to a photo-isomerable compound to form a monolayer over a nanopore interior and its surrounding surface. As with most undertakings based on abiotic nanopores, the nanopore is immersed in an ionic electrolyte with the ˜10 nm-diameter channel providing the only path for mass transport driven by a current along the ˜10 nm channel length. The approach is applicable to single-molecule DNA sequencing, where the electrophoresis of a DNA strand through a nanopore alters the ionic current flow to give rise to characteristic signals that can be used to recover the DNA base sequence. This paper is incorporated herein by reference.

Also known in the art are abiotic pores with silica substrates. An example is found in V. Zelenak, et al., “Photo-switchable nanoporous silica supports for controlled drug delivery,” New J. Chem., 2018, 42, 13263-13271. This paper describes the use of multiple coumarin molecules which are applied to the outside of a silica micropore where the coumarin molecules are driven to form a dimer using visible light. The dimerized coumarin traps a molecule of interest within the pore which can be released by irradiating the pore with ultraviolet (UV) light. However, this dimer-based system can only function for a small range of pore sizes having a characteristic size compatible with the employed dimers, and the dimer system described is said to function only on the exterior surface of the silicon pore.

Pore control in planar SiN substrates is described by Meller et al. (“Optoelectronic control of solid-state nanopores,” U.S. Pat. No. 10,613,076 B2, issued Apr. 7, 2020). Meller et al. aim to achieve pore control though purely optoelectronic means by manipulating the surface charges in the pore via light supplied by a laser, and they claim to be able to characterize an analyte by controlling the translocation speed of biopolymers, such as DNA, through these SiN pores.

I. Vlassiouk et al., in “Control of Nanopore Wetting by a Photochromic Spiropyran—a Light-Controlled Valve and Electrical Switch,” Nano Lett. 2006, 6(5), 1013-1017, claim to gate the entry of water into a pore by altering the hydrophobicity of spiropyran believed to be located on the surface of an alumina membrane. Vlassiouk et al. claim that the relevant structure acts as a photo-activated, non-selective “burst valve” providing a two-order-of-magnitude change in the flow of ionic or non-ionic fluids. When used with an ionic fluid, the device can function as a single use switch. However, the structure appears not to be reusable—being left in the “on” position—likely because the energy barrier to dewetting is too great.

T. Ma et al., in “Combining Light-Gated and pH-Responsive Nanopore Based on PEG-Spiropyran Functionalization,” Adv. Mater. Interfaces, 2018, 5, 1701051, describe the construction of relatively long pores whose interior surface has been functionalized by attaching a PEG molecule to a spiropyran bound to the pore interior. The Spiropyran provides light sensitivity, and the PEG provides a response to pH conditions. By varying the Spiropyran configuration and the pH value, the pore can be selective for anions under acidic conditions, and for cations at a neutral pH value. Thus, the pores could function as rectifying circuit components. However, there is no suggestion that Ma et al.'s devices can support a driven biomolecular transport, let alone providing selectivity over the process.

G. Laucirica et al., in “Redox-Driven Reversible Gating of Solid-State Nanochannels,” ACS Appl. Mater. Interfaces, 2019, 11, 30001-30009, show how a redox-sensitive coating external to a pore in a PET membrane can be used to control iontronic behavior at the entrance to a pore. Function in this device is related to the coating's electrostatic charge suitable for the control of non-specific ion species. Nevertheless, the approach is not susceptible to selectivity based on polarity or steric differences in analytes.

Remarkably different from the present disclosure, but also involving the ability to control the movement of small numbers of molecules through a membrane, is the method described by P. Li et al., in “Light-Driven ATP Transmembrane Transport Controlled by DNA,” Nanomachines. J. Am. Chem. Soc. 2018, 140, pp. 16048-16052. That paper describes how an ATP molecule can be driven across a membrane by irradiation through alternating light frequencies. This work shows how molecular motors can be used to move molecules across a membrane.

### BRIEF SUMMARY

In view of the various disadvantages exhibited in prior art systems, the present disclosure describes a new system for creating nanopore membranes first stabilized by a covalently bonded adhesion layer which are suitable for subsequent customized, reversible, and enhanced functionalization.

Providing reconfigurable functionality for nanopores would be of great value for both research and practical applications. For example, partitioning a heterogenous mixture of molecules into a set of homogeneous solutions for analytical or purification applications could be achieved by a first selective transport across a nanopore, reconfiguration of the nanopore and a second selective round of transport. A previous approach to reconfiguring nanopores is photo-isomerization of molecules bound to a nanopore's interior. The approach has the desirable feature that no chemical treatments are needed to change the functionality of the nanopore and the nanopore's structural changes are integral to the photochromophore. However, as a general proposition, achieving a full range of functionality would still require the use of different chemical structures.

In an aspect, the invention provides a method of processing silicon-nitride films into shelf-stable, functionalized nanopore devices. Features of the foregoing method include additional conventional steps to provide enhanced functionalization of the nanopore devices. Other features of the method include steps to reconfigure the nanopore devices to change the selectivity of transport through the nanopore. Yet another feature is the ability to employ steps to recycle the enhanced functionalized nanopore devices back to the original shelf-stable state so that a different enhancement can be made.

In another aspect, the present invention can provide shelf-stable nanopores which are subsequently both stabilized and functionalized by coating the nanopore interior. This coating can further serve as an adhesion layer for a secondary functionalizing step where specially designed chemical sites on the adhesion layer are configured to bind a variety of additional molecules chosen to provide desired further physical and chemical functionality. It is contemplated within the principles of the present invention to have a heterogeneous set of molecules serving as the adhesion layer. Altering the ratio of such different molecules present in the adhesion layer provides the ability to finely control more characteristics such as hydrophobicity, and also to offer initial control of binding specificity for targeted analytes.

A feature of the invention comprises providing nanopores made with a commonly available substrate such as silicon nitride where the adhesion layer is formed by covalently bonding organic molecules to the silicon nitride on the interior surface of the nanopore. In a feature, these organic molecules can be designed with one or more unsaturated terminal groups capable of bonding with the substrate through a reaction such as photo-hydrosilylation. After at least one unsaturated terminal group is bonded to the substrate, any additional unsaturated terminal group may remain unbound, providing a stabilizing effect to the substrate and modifying the wetting characteristics of the substrate. In another feature, the organic molecules are provided with chemical protecting groups that are designed to survive the conditions used for photo-hydrosilylation, and which protect reactive sites that will be employed in subsequent steps. In yet another feature, the subsequent removal of the protecting groups from the surface of the adhesion layer optionally exposes a carboxylic acid or other suitable functional group. And further steps, such as an esterification reaction, can then reversibly bond one or more molecules of a coupling partner which provides a secondary (enhanced) functionalized layer to the nanopores. A diverse range of secondary functionalization of the nanopore can thus be achieved using standard chemical approaches. The secondary functionalization provides another lever through which the selectivity (for example, binding an antigen or other molecule of biological significance) or gross properties (such as sterics or hydrophobicity) can be modified.

The availability of such a system would allow a practitioner to focus on the particular secondary functionalization needed for their work without having to master the entire process of nanopore fabrication, the stabilization of the nanopores and the nanopore substrate, or the chemistry of covalently binding organic molecules to silicon needed for initial (or “primary”) functionalization.

In a preferred embodiment, nanopores may be fabricated in aqueous conditions by controlled (dielectric) breakdown. These nanopores can then be subsequently coated within a fairly short period, e.g., about 30 minutes after pore formation, by photohydrosilylation with a custom-made DIOL molecule, leaving an —OH surface termination.

In one embodiment, the custom-made molecule 2,2-Di(2-propyn-1-yl)-1,3-propanediol (DIOL) is bound to solid-state SiNₓ nanopores fabricated by controlled dielectric breakdown to form a first (“primary”) adhesion layer which serves to both stabilize and partly functionalize the inner pore surface of the nanopores. Note that in this embodiment, each molecule in the adhesion layer provides two —OH terminal groups.

Characterizing the nanopores can be done by measuring their conductance under aqueous conditions through applying voltages and measuring currents to get I/V curves. In addition, detection of analytes can also be made by monitoring fluctuations in current flow which correspond to the translocation of molecules through the pores. Differences in the I/V characteristics obtained after further modifications of the nanopores can also be detected and exploited for analytical or other purposes.

For example, in one embodiment, treating the —OH groups with Adipoyl Chloride produces an ester which is easily hydrolyzed back into a carboxylic-acid-rich surface, and the carboxylic-acid-rich surface itself, with or without further treatment, provides functionalization that may be suitable for a desired application. Such carboxylic acid terminated nanopores, and more specifically the ester variants, are believed to be suitable for shelf-storage and to serve as off-the-shelf components providing a flexible platform for a diverse array of analytical and practical applications.

In a further feature, these carboxylic acid sites can, for example, be esterified with a molecule possessing desired size, charge, conformational, or other characters which can be used to customize the behavior of the pore further.

In a preferred feature, nanopores can be reconfigured reversibly. In one embodiment, pores coated with a carboxylic acid terminated inner pore surface, can have the terminal group cycling between a carboxylic acid, an amine, an ester, and then back to a carboxylic acid. This process allows the same setup to be preserved and reused.

In yet another feature, stabilization of nanopores can be accomplished by forming an adhesion layer with a homogeneous or heterogeneous mixture of stabilizing compounds.

In a further feature, secondary functionalization of nanopores can be accomplished with a homogeneous or heterogeneous mixture of coupling partner compounds.

In further embodiment, an original secondary functionalization layer can be modified by breaking internal bonds of a coupling partner compound and optionally performing subsequent additions of desired functional groups, thereby modifying an existing secondary functionalization layer.

### BRIEF DESCRIPTION OF FIGURES

The patent or application file contains at least one drawing executed in color. Copies of this patent or patent application publication with color drawing(s) will be provided by the Office upon request and payment of the necessary fee.

To easily identify the discussion of any particular element or act, the most significant digit or digits in a reference number refer to the figure number in which that element is first introduced. The patent or application file contains at least one drawing executed in color. Copies of this patent or patent application publication with color drawings will be provided by the Office upon request and payment of the necessary fee. The present disclosure may be better understood with reference to the following figures.

FIG. 1A and FIG. 1B illustrate the overall molecular-scale scheme of a reconfigurable nanopore according to the present disclosure.

FIG. 2 illustrates the custom molecule, 2,2-Di(2-propyn-1-yl)-1,3-propanediol useful for the present invention.

FIG. 3 illustrates the synthesis of the custom diol molecule as used in an embodiment of the present invention.

FIG. 4 illustrates the overall scheme of a custom functionalization chamber for practicing the present invention.

FIG. 5 is a photograph of the assembled functionalization chamber according to principles illustrated in FIG. 4.

FIG. 6 is a photograph of the disassembled functionalization chamber of FIG. 5 with component details.

FIG. 7 illustrates primary functionalization of a nanopore using a custom molecule according to an embodiment of the present invention.

FIG. 8A illustrates a perspective overview typical analytical cell from the prior art, with a nanopore containing membrane and reservoirs.

FIG. 8B illustrates a vertical cross-section of a typical analytical cell from the prior art showing the connections between a nanopore containing membrane and reservoirs.

FIG. 9 shows, according to an embodiment of the present invention, secondary functionalization by converting an alcohol to an ester.

FIG. 10A, FIG. 10B, FIG. 10C, FIG. 10D, and FIG. 10E show nanopore conduction and organic molecule transport changes upon interconversion of an ester to an alcohol and back to an alcohol, according to an embodiment of the present invention. FIG. 10A shows and ester form. FIG. 10B shows a diol. FIG. 10C shows a recreated ester form. FIG. 10D shows the sensing of DNA. FIG. 10E shows the sensing of maltodextrin.

FIG. 11A, FIG. 11B, and FIG. 11C each show for three independent nanopores, according to an embodiment of the present invention, conventional conductance measurements processing steps. For each of these figures, point 1 show the as-formed (by controlled dielectric breakdown) conductance. Point 2 shows conductance after coating with the 2,2-di(2-propyn-1-yl)-1,3-propanediol by photohydrosilylation. Finally, Point 3, shows conductance after subsequent treatment of the coated pore with adipoyl chloride. Each of the independent nanopores behaves as expected.

FIG. 12A, FIG. 12B, and FIG. 12C characterize DNA translocation through a diol functionalized pore showing functional modification of the pore. FIG. 12A is a current trace. FIG. 12B shows event data for DNA. FIG. 12C provides a histogram of correlated conductance.

FIG. 13A, FIG. 13B, and FIG. 13C characterize DNA translocation through pore after secondary functionalization of a diol functionalized pore with adipoyl chloride showing further functional modification of the pore. FIG. 13A is a current trace. FIG. 13B shows event data for DNA. FIG. 13C is a histogram conductance for all events.

FIG. 14 shows conductance measurements which show an embodiment of the present invention supporting reversible formation of calcium acetate at the carboxylic acid terminations of the functionalized pore.

FIG. 15 shows additional conductance measurements of calcium acetate formation and dissociation at the carboxylic acid terminations of the functionalized pore further illustrating the reusability of a functionalized pore.

FIG. 16 illustrates Nanopore I/V curves for a SiNₓ nanopore at differing stages of processing which shows how functional enhancement progresses in an embodiment of the present invention.

FIG. 17 shows exemplary variations in secondary functionalization compatible with the custom diol illustrating the diversity that can be achieved according to the present invention.

FIG. 18 shows an exemplary amine based stabilizing compound suitable to apply the present invention where a non-rigid functional group is desired.

FIG. 19 shows an exemplary cyclic ether based stabilizing compound suitable to apply the present invention where a rigid functional group is desired.

FIG. 20 shows an exemplary aromatic amine based stabilizing compound suitable to apply the present invention where aromatic interaction with an analyte is desired.

FIG. 21 shows a stabilizing compound capable of crosslinking with the same or other compounds to create a durable functional coating.

FIG. 22 illustrates pore conductance at various steps of the reversible functionalization of a carboxylic-acid-rich adhesion layer showing how an embodiment of the present invention can predictably cycle through alterations in a pore designed to detect different analytes.

FIG. 23 illustrates the protection of a stabilizing compound's coupling site prior to the forming of an adhesion layer.

FIG. 24 illustrates the alteration of a coupling partner on a previously created secondary functionalization layer without replacing the coupling partner.

### DETAILED DESCRIPTION

Unless otherwise noted, technical terms are used according to conventional usage.

As used in the specification and claims, the singular form “a”, “an”, or “the” includes plural references unless the context clearly dictates otherwise. For example, the term “a nanopore” includes a plurality of nanopores including mixtures thereof.

When a dimensional measurement is given for a part herein, the value is, unless explicitly stated or clear from the context, meant to describe an average for a necessary portion of the part, i.e., an average for the portion of the part that is needed for the stated purpose or function. Any accessory or excessive portion not necessary for the stated function is not meant to be included in the calculation of the value.

As used herein, the approximate symbol, i.e., “˜”, unless otherwise indicated, indicates that the discussed value is equal to the indicated value plus or minus 5% of the indicated value. As an illustration, if the test refers to “100±5%” the indicated value may range from 95 to 105.

As used herein, the term “about” means within plus or minus 10%. For example, “about 1” means “0.9 to 1.1” and so on.

As used herein, the term “adhesion layer” refers to the surface formed by a stabilizing compound which has been engineered to feature coupling sites which are exposed functional groups capable of bonding to members of a desired set of compounds or molecules serving as a coupling partner. For example, an adhesion layer containing alcohol groups can be easily reacted with an acyl chloride to form an ester, which may be useful for nanoscale applications. Note that an adhesion layer itself, such as one terminated with the original alcohol or the esterified form as above, may provide desired functionality without taking further steps to prepare the adhesion layer for binding to a coupling partner. However, assuming the intent is to prepare for coupling partners with the prior example, an adhesion layer containing ester coupling sites can, through hydrolysis, be converted to carboxylic acid coupling sites which can be readily (and reversibly) bound to positive ions or coupled to a variety of coupling partners to provide a range of nanopore functional modifications.

As used herein, the term “analysis cell” refers to an apparatus configurable to hold and processes one or more fluidic samples employing a nanopore-containing membrane between at least two fluid reservoirs. An analysis cell may be used as an environment for various purposes such as identifying, controlling, purifying, or modifying a chemical or a solution of chemicals.

As used herein, the term “analyte” refers broadly to chemicals measured, identified, controlled, purified, or modified by a device or system according to the current disclosure.

As used herein, the term “biopolymer” refers to compounds that are important to biological and biochemical processes and are multi-unit compounds made up of monomeric units. Generally, biopolymers are degradable and biocompatible. Examples of biopolymers are oligo-or polynucleotides (e.g., DNA, RNA), oligo-or polypeptides (e.g., protein, shorter oligopeptides, collagen, actin, and fibrin), and oligo-or polysaccharides which are linear or branched chains of carbohydrates (e.g., starch, cellulose, alginate, sugars, charged or neutral carbohydrates, etc.). Lipids, which have extended carbon chains can, for the purposes of practicing the disclosed invention, be grouped with biopolymers. Biopolymers can also consist of multiple different monomer types, such as glycoproteins and glycolipids consisting of sugar-decorated proteins and lipids, respectively. There are other polymers that are not biopolymers, e.g., petroleum-derived polymers which are widely used in industrial applications such as plastic-making.

As used herein, the term “bonding site” refers to a location on an organic molecule configured to controllably form a covalent, ionic bond, or physisorption with a silicon-rich substrate when the organic molecule is to function as a stabilizing or adhesion layer. In a preferred embodiment, a bonding site comprises a carbon atom proximate to an unsaturated carbon-carbon bond. In the present disclosure, it is not expected that all available bonding sites need to be bound to the substrate for a given nanopore configuration. It may, however, be desirable to achieve multiple bonds to a substrate to increase the adhesion layer's stability.

As used herein, the term “cell” refers to an apparatus that holds and processes one or more fluidic samples employing a nanopore-containing membrane between at least two fluid reservoirs.

As used herein, the term “clog” (or “clogging”) refers to where a nanopore's properties have been modified because a molecule type has been situated, either reversibly or permanently, on, near, or within a nanopore such that the ability of the nanopore to interact with some molecule type has been changed. Generally, clogging means that the nanopore's ability to transport some molecule type through pore has been diminished, but a diminishment in the nanopore's ability to distinguish between two molecule types would also be considered clogging. In general, the indication of a clog is an undesirable change in what should be an open-pore current or current noise levels measured with respect to a nanopore.

As used herein “configuration,” (or “configurational”) with respect to a molecule, denotes either a particular geometry, diastereomer, or isomer of a molecule distinguishable from another geometry with regard to its influence on any property of a nanopore.

As used herein, “coupling partner” refers to a compound capable of being bound to an adhesion layer to provide changed or enhanced functional activity in connection with the use of a nanopore. In effect, a coupling partner forms a new surface over the adhesion layer of a nanopore which has been modified by a stabilizing compound. A coupling partner may be chosen to provide overall changes to pore functions, e.g., by altering the effective pore size through gross changes in steric interaction or hydrophobicity. A coupling partner may also be chosen for the purpose of providing a specific desired interaction with a chosen analyte. A sampling of the range of conventional organic compounds that can be coupling partner for practicing the present invention include commercially marketed “connecting molecules,” e.g., compounds as those currently offered by MilliporeSigma (emdmillipore.com/US/en/fascinated-by-analytics/Connecting-Molecules/mkWb.qB.a.kAAAFEyoUwckZ7,nav). Non-limiting examples include n-propanephosphonic acid anhydride, various bifunctional boronic acids, oxo-acids, vinylphosphonic acids, chiral cyclopentenol, nicotinic acids and derivatives, aliphatic amines, and various heterocycles.

As used herein, the term “coupling site” refers to a location on a stabilizing compound capable of binding a coupling partner through chemical reactions and, optionally, further capable of site-reset though subsequent chemical reactions. In an exemplary embodiment, a coupling site is a carboxylic acid's terminating hydroxyl group which can be transformed into an ester with a selected coupling partner, and then returned (“site-reset”) to a terminal hydroxyl group through a second set of chemical reactions.

As used herein, the term “freshly formed,” or “nascent” refers to the state of a surface region that is void of significant oxide formation or other masking groups that would otherwise preclude substantive and intended chemical attachment to the underlying silicon or nitrogen atom(s). Accordingly, a “freshly formed” silicon surface or region has available silicon at sufficient density for chemical attachment. Similarly, a “freshly formed” silicon nitride surface or region has available amine (Si—N—H₂) at sufficient density for chemical attachment (e.g., by forming covalent Si—N—C bonds on the surface or region). Some oxide formation on a “freshly formed” silicon or silicon nitride surface/region is allowed within the meaning of the present invention as long as the intended chemical attachment or functionalization reaction can proceed and result in successful and significant surface modification. In a preferred embodiment, a “freshly formed” silicon or silicon nitride surface/region is provided without the use of any etching (e.g., hydrofluoric acid) treatment to strip oxides on the surface or region-such a surface may be provided directly through the nanopore fabrication technique called “dielectric breakdown.” If the intent is to stabilize a pore through means other than hydrosilylation, such as stabilizing with silanes or silatranes, then an oxidized surface, rather than a freshly formed surface would be preferred to form an adhesion layer.

As used herein, “functional enhancement layer” refers to the surface formed by a coupling partner compound bound to an exposed stabilizing compound of an adhesion layer via a coupling site. In an exemplary embodiment, the functional enhancement layer may consist of a compound such as diaminopropane, which, when coupled to the adhesion layer, substantially changes the translocation of molecules through a nanopore. A wide range of compounds can provide desirable functional enhancements for practicing the present invention. A suitable choice of coupling partner can enhance a useful property of a nanopore interior surface, such as hydrophobicity, surface charge polarity, dependence on pH, or steric interference. Any of these changes might affect translocation with varying levels of specificity with respect to a given analyte.

As used herein, “functionalization” refers to the modification of a surface achieved by binding of a molecule or element bound to a surface which imparts a desired property to the surface which alters the behavior of the surface with respect to a chemical process occurring within a characteristic distance from the surface under a specified set of conditions.

As used herein, the term “functionalization chamber” refers to an apparatus configurable to hold a membrane or film usually containing one or more nanopores, in contact with reagents and which is configured to provide conditions supporting a desired set of reactions. For example, a window to allow irradiation of the membrane or film, such as that employed according to the present disclosure, may be provided. Such a chamber may be used according to the present disclosure, to facilitate binding of an adhesion layer to a membrane or film. In some embodiments, the reactants are in solution, however, the use of solid reagents or pure reagent may be preferred.

As used herein, “functionalized pore” refers to a nanopore coated with at least a partial layer of bound molecules which alter how the pore surface interacts with analytes (other molecule types or ions). These bound molecules may be further functionalized by subsequent chemical modifications. Typically, such secondary functionalization is accomplished by modifying a terminal portion of the bound molecule.

As used herein, “membrane or film” refers to a material having a thickness between about 0.5 to 100 nm. A typical membrane or film fabricated from a material such as silicon nitride will be specified to a thickness with a tolerance such as ±5%. In a typical embodiment, the film is mounted in a frame having dimensions on sub-centimeter scale, but which frame is big enough to be handled directly by a technician. The mounted membrane or film is suitable for processing to create nanopores. When clear by context, the term membrane or film is meant to include any relevant nanostructures created in accordance with this disclosure, to wit, nanopores.

As used herein, “molecular type” refers to a chemical species, nanoparticle, nanoscale entity, or compound in its molecular state or ionic state (possibly soluble in a chosen fluid) which can be distinguished from another species or compound using an intrinsic physical or chemical property. For example, charged molecular types could be distinguished from uncharged molecular types by employing a mechanism based on charge, or a large molecule type might be distinguished from small molecule type by a mechanical process. A molecule type might refer to a stereo-isomer of the same chemical compound because the different isomers could be distinguished from one another by one type's affinity differing from another type's affinity to a third chiral chemical. A molecule type also might be distinguished from another type by molecular configuration.

As used herein, “nanopore” refers, in one form, to a hole or opening in a membrane or film of a diameter between about 0.5 nm and about 100 nm which fully penetrates the membrane or film. It is not required that the membrane or film be perfectly flat-a membrane or film having significant overall curvature is sufficient to define a nanopore provided that the local curvature of the membrane or film within a distance equal to one or more diameters of the opening does not exceed the radius of the opening. In other words, as long as the membrane or film is not so curved that it folds back on itself within a diameter of the opening, then the nanopore is well defined. This accounts for nanopores which are fabricated in other than conventionally flat membranes or films, such as folded structures optimized to maximize the effective area of a device which is packaged in a given volume. In another important form, a nanopore may be fabricated in the form of a nanopipette which is suitable for sensing applications. At the scales relevant to these nanopores, the behavior of liquids can change, and the relative effect of structural or electronic changes to the atoms or molecules interior to the nanopore may increase dramatically. These interfacial properties of the nanopore are long-scale relative to the pore's cross-section, and provide the potential to fully and dynamically control mass transport with single molecule precision.

The term “opening” refers to a penetration through a substrate defining the gross geometry of a nanopore. A nanopore is usually more than a simple opening in a substrate in as much as a nanopore, as preferably employed herein, is functionalized.

The term “photo-isomer” herein refers to molecular configurations achieved by radiating a photo-responsive molecule with light radiation, e.g., with wavelength in the visible (to human) or ultraviolet range. In preferred cases, exposure to light of a chosen wavelength can cause a molecule to predictably and stably assume a particular configuration. In some cases, subsequent exposure of the same molecule to the same or different radiation can convert the molecule to a different configuration. Note that as used herein, photo-isomers can, in some cases, act similarly to chemical isomers, where molecules of the same empirical formula vary by bond topology. In other cases, photo-isomers retain their bond topology, and only the geometrical configuration of the molecule changes as a result of changes in the orientation of a part or parts of the molecules. Photo-isomers can be quite stable, e.g., by holding a configuration for half-lives as long as days at room temperature. Also significant is that properties that photo-isomers have in bulk generally can be retained when photo-isomers are constrained to nanopore scale. These facts allow photo-isomers to form the basis of reversibly configurable nanopores when photo-isomerable molecules are bound to a nanopore's interior. See “configuration” above.

As used herein, “photo-isomerable” refers to a molecule being capable of assuming different photo-isomers.

“Photohydrosilylation” refers to the use of light-induced hydrosilylation to bind an organic compound to a silicon rich substrate. See Dwyer et al. (“COVALENT CHEMICAL SURFACE MODIFICATION OF SURFACES WITH AVAILABLE SILICON OR NITROGEN,” U.S. Pat. No. 10,519,035 B1, issued Dec. 31, 2019).

As used herein, “photoswitch” refers to the act of converting a photo-isomerable molecule between various configurations of its photo-isomers.

As used herein, the terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded.

As used herein, “pore property” or “pore properties” refers to physical properties of a nanopore which are significant at the nanoscale. These properties are diverse, including (but not limited to) pore diameter, pore thickness, molecular polarity, hydrophobicity of a coating, surface coating, base material (substrate), charge characteristics, steric properties of molecules bound to the interior of the pore, the response of a surface material to pH, charge, solvent, temperature, electrolytes, analytes, the voltage applied to or the current carried through the pore. A specific set of pore properties will allow different molecular types to pass through the pore while rejecting other molecular types.

As used herein, “pore-voltage-gradient” refers to the profile, as a function typically defined along the axis defined along the pore entrance to the pore exit, of a voltage potential. The potential is frequently applied externally with the goal of powering active transport of solvent or molecular types through a pore.

As used herein, “proximate” to a first location refers to a second location on a molecule which is immediately adjacent to or (on average) close enough, either by primary structure or by virtue of a molecular conformation or folding, such that a meaningful interaction can occur between the first and second locations.

As used herein, “resistive-pulse sensing” refers to a change in measured voltage or current across a pore in which each pulse corresponds to the passage of one or more molecules though a pore. For resistive-pulse sensing to work well, the baseline noise level must be low enough to have sufficient signal to noise ratio, plus the pore properties must be reasonably stable over the measurement period.

As used herein, the term “shelf-stable” refers to a nanopore characteristically being able to be stored for a week or more at room-temperature or otherwise without refrigeration and under typical sea-level air pressure or, more generally, in an ambient condition, or with further stabilization by controlling surrounding temperature and atmosphere (e.g. cooling and removing oxygen) or by the addition of water, aqueous electrolyte, or organic solvent to the storage container, so that it is able to be stored for approximately 6 months or more while retaining functionality for an intended use. Shelf-stable preferably includes sufficient stability to tolerate elevated temperature for a period of time needed for shipping the nanopore to another location.

As used herein, the term “silicon nitride” refers to any chemical compound consisting substantially of two elements only: silicon and nitrogen, such that it can be chemically represented as SiₓN_{y} or SiNₓ for simplicity. Though SiₓN_{y} exists with differing ratios of Si to N, the stoichiometric form is Si₃N₄. In contrast, a “silicon-rich” silicon nitride, as used herein, refers to a silicon nitride (SiₓN_{y}) where the ratio of “x” over “y” is greater than 0.75, i.e., x:y>3:4, e.g., where “y” is 4, “x” can be 4, 5, 6, 8, or more. Other examples of “silicon-rich” Si/N ratio in silicon nitride include: 0.77, 0.82, 1.02, 0.95, 1.14, 0.87, and so on (see Journal of Vacuum Science & Technology A: Vacuum, Surfaces, and Films 14, 2879 (1996)). A silicon nitride substrate can be one or more layers of silicon nitride deposited on a semiconductor (e.g., silicon) base.

As used herein, “site-reset” refers to any process whereby a site on a selected chemical compound is returned to an original or prior chemical state after having been bound to a coupling partner. The term site-reset is chosen to distinguish between a situation where a chemical site may be modified in a conventionally reversible sense wherein reactants and products are in an equilibrium. In contrast, site-reset may require chemical reactions consuming significant energy, or changes of conditions, to remove a coupling partner and may proceed through entirely different chemical mechanisms than the mechanism used to couple the partner to the site in its original or prior state.

As used herein, “stabilizing compound” refers to an organic compound configured to be capable of binding to a substrate (typically to form an adhesion layer) through one or more covalent bonds at one or more bonding sites on the stabilizing compound. When the stabilizing compound is bonded after nanopore fabrication, the stabilizing compound also binds to the nanopore interior. In a preferred embodiment employing a silicon rich substrate, the stabilizing compound is bound to the nanopore interior through hydrosilylation. The hydrosilylation reaction path is enabled by configuring the stabilizing compound to comprise an unsaturated terminal region. In some embodiments, the hydrosilylation is driven by light, as a photohydrosilylation reaction. More than one such terminal region may be engineered so that multiple bonding sites of the stabilizing compound are available to bind to the substrate. When bound to a substrate, a stabilizing compound can passivate an otherwise reactive surface. For example, in conjunction with a substrate such as silicon nitride which is susceptible to hydrolysis, an organic stabilizing compound can add substantial hydrophobicity or physical coverage to the substrate surface, inhibiting dissolution, hydrolysis, and oxidation. In addition to stability, the adhesion layer formed by the stabilizing compound may also provide desired function to the substrate. In the present disclosure, the bound stabilizing compound may also provide an adhesion layer which includes a coupling site to bind a coupling partner that can provide enhanced functionality.

As used herein, being “stable” refers to a nanopore which: (1) does not, as originally fabricated or subsequently stabilized with a stabilizing compound, clog during a suitable operational period, e.g., more than 30 minutes, an hour, a day, etc., (2) does not clog during a suitable operational period under conditions where control has been achieved by altering the pore's properties caused by the addition of a stabilizing compound (or other chemical changes) and the pore exhibits a current flow under an applied pore-voltage-gradient such that a measurable signal-to-noise ratio can be achieved for resistive-pulse sensing, or (3) is measurably unchanged in its physical dimensions or conductance over a desired period of time, e.g., 30 minutes, an hour, a day, a week, and so on. A suitable or desirable period for the purpose of being stable herein is preferably more than a day, more preferably, more than a week.

As used herein, “substrate” is a term to generally refer to the membrane or film used as a starting material for the creation of nanopores and generally encompassing the resulting active portion of nanopore based system.

As used herein, “translocation” refers to the transport of a molecular type from one side of a nanopore through and out of the nanopore to the other side. The motive force could be hydrostatic pressure, diffusion, osmosis, electroosmosis, electrophoresis, or a combination of these. Other than pumping applications where the goal is simply changing concentrations or mass transport, a set of translocations can function to sort, separate, or isolate, molecular types from one another. In the case of characterizing or discriminating, the event of translocation can function as a “read” operation to extract information from molecular type. Read operations on genetically encoded molecules could enable extremely rapid genome sequencing or be applied to proteomic systems. Read operations on molecular types from certain families of chemicals, such as carbohydrates, could enable extraordinarily dense data storage and retrieval technologies.

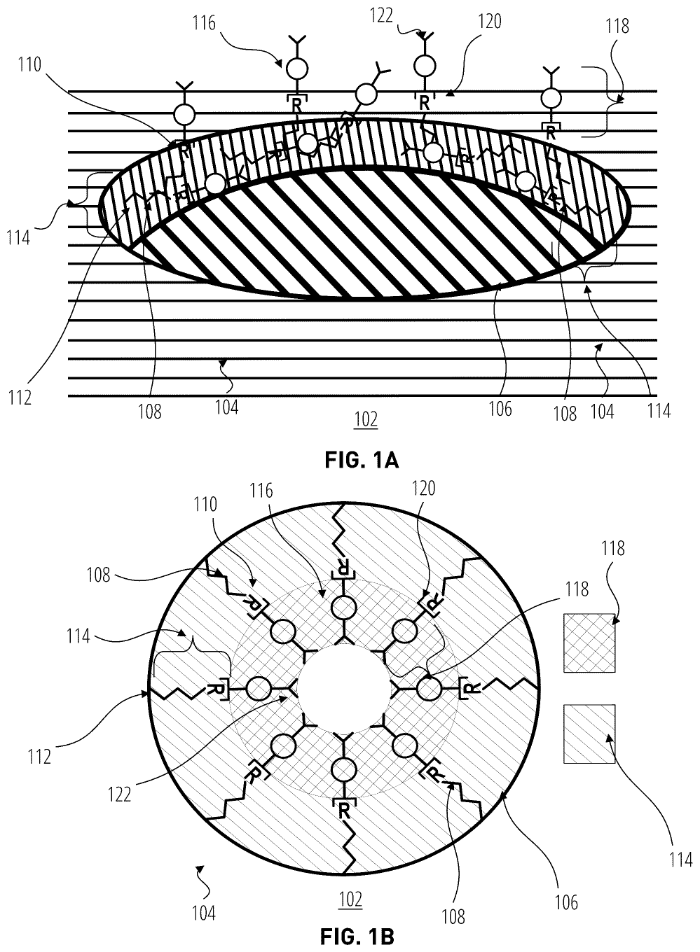

The overall scheme 102 of a reconfigurable nanopore according to the present disclosure are schematically illustrated in FIG. 1A and FIG. 1B, which show the principal components of the scheme in relation to one another. FIG. 1A provides a 3D view of a pore in accordance with the scheme, and FIG. 1B shows a top-down view of the scheme. The hatched area (labeled in the legend box as 114) represents an adhesion layer. The cross-hatched area (labeled in the legend box as 118) represents a functional enhancement layer. As the scheme is implemented at nano-scale, neither FIG. 1A nor FIG. 1B are meant to convey any sense of scale.

Referring to FIG. 1A and FIG. 1B, a membrane or film 104 containing one or more recently fabricated nanopores 106 is bonded to a stabilizing compound 108 at the compound's bonding site 112. In aggregate, the population of bonded stabilizing compound 108 molecules form an adhesion layer 114. This adhesion layer 114 generally serves to both passivate and to provide primary functionalization of the surface of the membrane or film, including the interiors of any nanopores 106. Subsequently at a convenient time, a selected coupling partner 116 is bound to a coupling site 110 on the adhesion layer 114. The coupling partner 116 is chosen to enhance the functionality of the surface of the membrane or film 104 generally, and the nanopores 106 surface within the membrane or film. The coupling partner 116 includes a coupling bonding site 120 designed to readily bond to the stabilizing compound 108's coupling site 110. The coupling partner 116 also includes a functional termination 122 providing desired binding or other characteristics to the pore. In aggregate, the population of bonded coupling partner 116 molecules form a functional enhancement layer 118.

The chemical properties of the coupling site 110 and the coupling partner 116 are preferably selected or synthesized to allow the coupling partner 116 to be later chemically removed from the coupling site 110 to make way for a different coupling partner 116 to access the coupling site 110 and be bound therethrough so that a change in function of the nanopores 106 is achieved. A potential variation from the foregoing is to select a coupling partner 116 having an internal bond that can be broken under different conditions than those used to break the bond between the coupling bonding site 120 and the coupling site 110. Doing so enables a different method for modifying the functional enhancement layer 118 and cycling through different functional terminations 122.

In an exemplary embodiment, nanopores 106 were fabricated by controlled dielectric breakdown in 15±2 nm thick SiNₓ membranes in 1 M KCl, 10 mM HEPES solutions at pH 7, as described previously by Dwyer. At this point, the SiNₓ surfaces remained highly susceptible to degradation by oxidation and hydrolysis, necessitating careful handling and prompt subsequent processing.

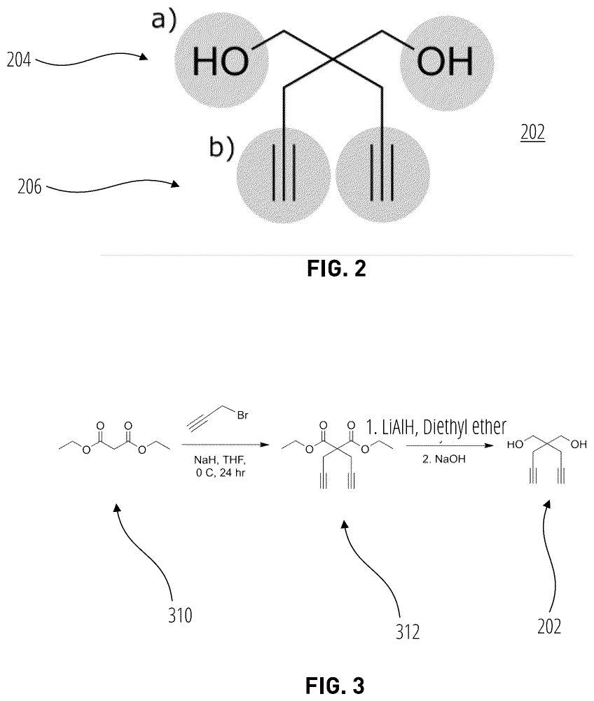

In this example, the raw nanopores were coated within 30 minutes of formation by photohydrosilylation with the custom DIOL 202 molecule of FIG. 2, which functions as the stabilizing compound 108, leaving an —OH surface termination, as shown in FIG. 3. This coating constitutes the adhesion layer 114, which both stabilizes and chemically functionalizes the nanopores 106. The details of the custom 2,2-Di(2-propyn-1-yl)-1,3-propanediol are shown in FIG. 2. The molecule features a hydroxyl bonding site 204, and a reactive alkyne or alkene region 206, both of which are used in reactions to achieve the overall reconfigurable nanopore scheme 102.

In an embodiment the custom DIOL 202 molecule was synthesized by the steps shown in FIG. 3. The starting material is the symmetrical dual ester 310 as shown at the left of the reaction sequence. Propargyl surface groups, meant to provide the reactive alkyne region 206, were added by supplying propargyl bromide. Finally, employing chemistry well known in the art, subsequent carbonyl to hydroxyl reduction was achieved with a lithium aluminum hydride reduction performed in diethyl ether. (See: J. Am. Chem. Soc. 2014, 136, 6, 2538-2545). As synthesized, the custom DIOL 202 molecule is an example of a stabilizing compound 108 suitable for bonding to the membrane or film in a functionalization chamber, according to principles of the present invention.

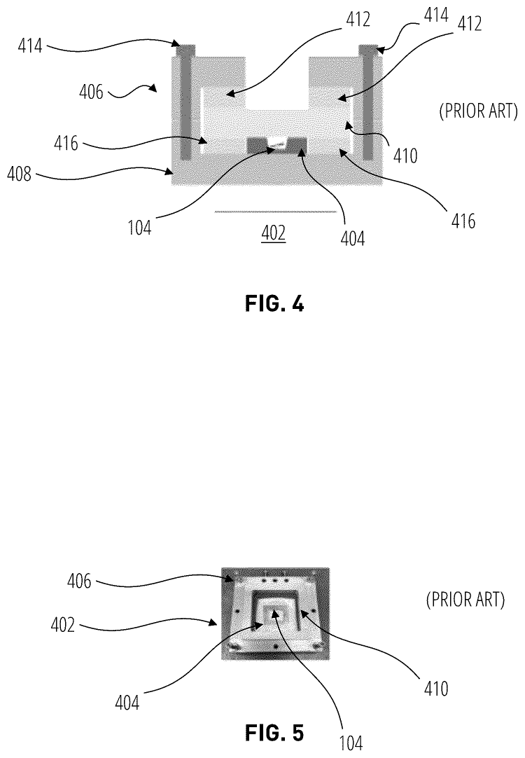

With a stabilizing compound 108 ready, the next step in fabricating the reconfigurable nanopore is to create an adhesion layer 114. This is typically accomplished by bonding the stabilizing compound to the membrane or film 104 in a functionalization chamber. Referring now to FIG. 4, an exemplary functionalization chamber 402, as known in the art, is shown and may be used to in accordance with the present disclosure to bond an organic compound to a nanopore-containing membrane or film. This organic compound will provide an adhesion layer which passivates the membrane or film's surface and provides for an initial functionalization of the surfaces of the membrane or film and any nanopore disposed in the membrane or film. This same chamber can be used for subsequent enhanced functionalization through coupling partner molecules. As described above, suitable nanopores may be fabricated through dielectric breakdown, or other techniques. The role of the functionalization chamber is to provide a controlled environment where an untreated nanopore containing membrane or film can be immersed in solvated desired reactants, where solvent evaporation is controlled by containing the solution in a closed vessel, and (in a preferred embodiment) allowing for the irradiation of the membrane or film with appropriate UV light to drive binding of an adhesion layer to the membrane or film through a photohydrosilyation reaction.

The overall scheme of the custom functionalization chamber 402 employed in a preferred embodiment, is as follows: A nanopore containing membrane or film 104 is mounted on a custom PTFE reaction well 404. A quartz cover plate 410 is sealed above by a top silicone rubber gasket 412 against a top chamber frame 406. The quartz cover plate 410 is sealed to the reaction well 404 using a bottom ePTFE gasket 416. The bottom chamber frame 408 is also sealed to the bottom ePTFE gasket 416 positioning the functionalization well 404 approximately centered under the quartz cover plate 410. The top chamber frame 406 is rigidly connected to the bottom chamber frame 408 with retaining screws 414. The top chamber frame 406 and bottom chamber frame 408 are, in this example, machined aluminum and the small functionalization well 404 was hand made from a block of PTFE.

For further clarity, a top-view photograph of the assembled functionalization chamber 402 is shown in FIG. 5. This photograph shows how the functionalization well 404 containing the membrane or film 104 is protected from the atmosphere while remaining accessible to be irradiated from above. The functionalization well 404 can be filled with reactants as desired to modify the membrane or film 104.

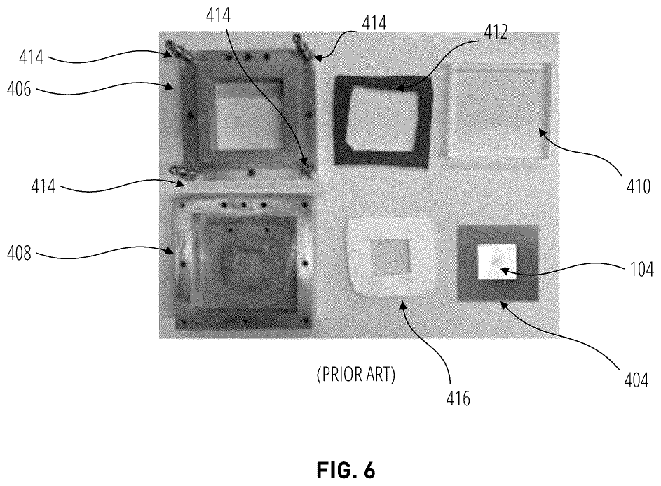

The various components of the functionalization chamber can be assembled with ordinary laboratory tools, and make use of typical materials used for chemical experimentation. The chamber is easily disassembled for cleaning and reloading with a nanopore containing membrane or film. Referring to FIG. 6, the set of disassembled parts comprising a typical functionalization chamber 402 can be readily handled by a technician without the need for special tools. The lower right-hand side of the photograph shows an overlay of a zoomed-in portion of the custom PTFE functionalization well 404, in which the membrane or film 104 containing nanopores 106 is placed for functionalization.

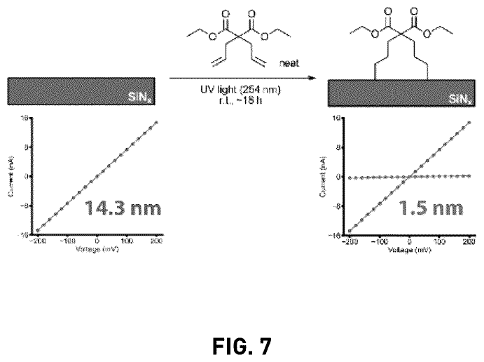

FIG. 7 illustrates the result of a photohydrosilyation reaction of a molecule similar to the custom DIOL 202 with SiNₓ. The molecule differs from the DIOL 202 in having only a double bond (as opposed to a triple bond) which will subsequently bond to the SiNₓ and also that it is in an ester form. The effective pore size is altered as a result of the reaction. A nearly identical reaction was used with the DIOL 202 molecule, showing through these representative reactions that primary functionalization of a nanopore not only can be achieved using the custom molecule of FIG. 2, but also with related variants. The resulting nanopore 106 can be used as is, or enhanced functionalization can be achieved with further steps in the functionalization chamber or in an analysis cell.

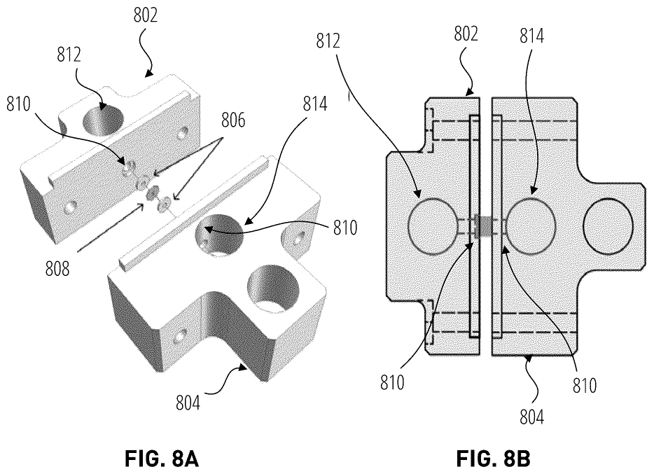

For example, an analysis cell, such as that employed in the art, is shown in FIGS. 8A and 8B, mounting a thin-film nanopore that provides source and target reservoirs suitable for active or passive transport of analytes and the detection of single molecules. The cell's overall structure is made from a left cell frame 802 bolted to a right cell frame 804 sandwiching a nanopore chip 808 between the frames and nanopore chip gaskets 806. A source reservoir 812 communicates with fluid channel 810, allowing fluid contact with a nanopore chip 808. In turn, the nanopore chip 808 communicates with a target reservoir 814 through a fluid channel 810. Conventional additions, such as valves, removable chambers, fluid and electrical attachments are known in the art and employed for convenience in any given application.

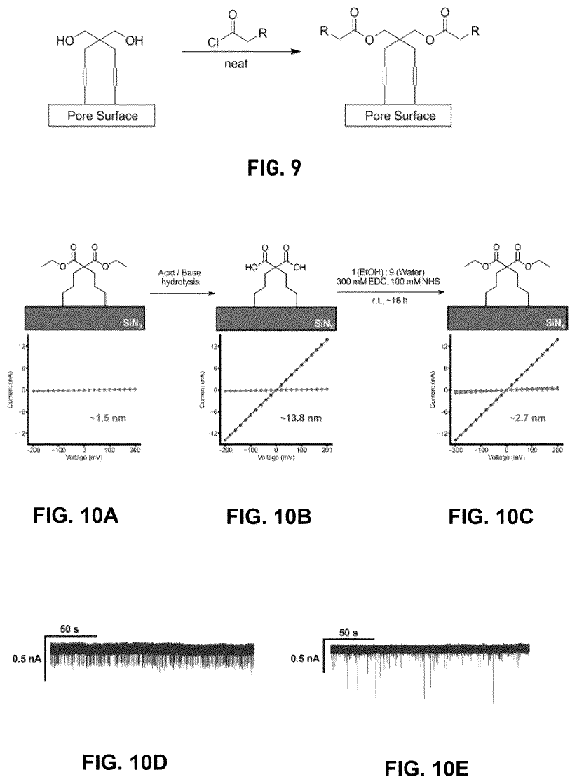

FIG. 9 illustrates an example of further functionalization; using a straightforward reaction, acyl chloride addition can be done in neat adipoyl chloride. The adipoyl chloride reacts with the hydroxyls of the custom DIOL 202 molecule on the surface of the nanopore, creating an ester. Upon hydrolysis, a new carboxylic acid terminated inner pore surface results. The completion of this step in this exemplary embodiment, creates the initial passivation and primary functionalization of the membrane or film 104 and the nanopores 106 within it. At this point, the membrane or film and its nanopores have been stabilized to the point where the structure can be stored for extended periods of time without the precautions that would be necessary to avoid typical degradation of bare SiNₓ nanopores. In other words, the surfaces are now stable, and preferably, shelf-stable.

The custom DIOL 202 is but one example of a stabilizing compound 108 that can be used to create a stable adhesion layer 114. Moreover, there is no requirement or limitation for using only a single species of stabilizing compound 108. The use of a mixture of stabilizing compounds 108 would provide the ability to alter both the primary functionalization and passivation properties. The passivated nanopores 106 are then suitable for use with analytes.

The nanopores processed according to principles of the present invention are also suitable for interconversions which can vary the secondary functionalization. FIG. 10 shows interconversion between an ester form of the custom stabilizing compound to a carboxylic acid form and back via an acid/base hydrolysis cycle. The charts below each step show the corresponding change in conductance of the pore. FIG. 10A illustrates the ester form, FIG. 10B the carboxylic acid form, and FIG. 10C the restored ester form. FIG. 10D shows that sensing protocols conducted in 1M KCl buffered to pH 7 by 10 mM HEPES using the hydrophilic pore could sense both DNA (3 kb dsDNA), and FIG. 10E shows the sensing of the complex carbohydrate maltodextrin under similar conditions.

To further confirm functionalization and functionality of the modifications on the nanopore surface in the example, several experiments were carried out. FIG. 11 shows conductance measurements of a nanopore at successive processing steps. The diameter of all pores were calculated from the conductance observed through I/V measurements. The diameter calculations demonstrated a reduction in pore size as each layer was added to the nanopores in all cases (a-c) in FIG. 11. The diameter was determined by taking the linear fit of the I/V data and taking the slope, or conductance, and plugging it into Equation 1 (below).

FIG. 11 shows conductance measurements of three different pores as each new coating was added to the pores. The gross effect of the coatings is clear in as much as conductance decreases after each coating is applied. In particular, conductance measurements after 1) initial controlled dielectric breakdown fabrication, 2) photohydrosilylation with DIOL, and 3) secondary functionalization with adipoyl chloride, are shown as in each of the three examples of FIG. 11. The data points are an average of three I/V measurements at each stage while the error bars are the error across each three-measurement average.

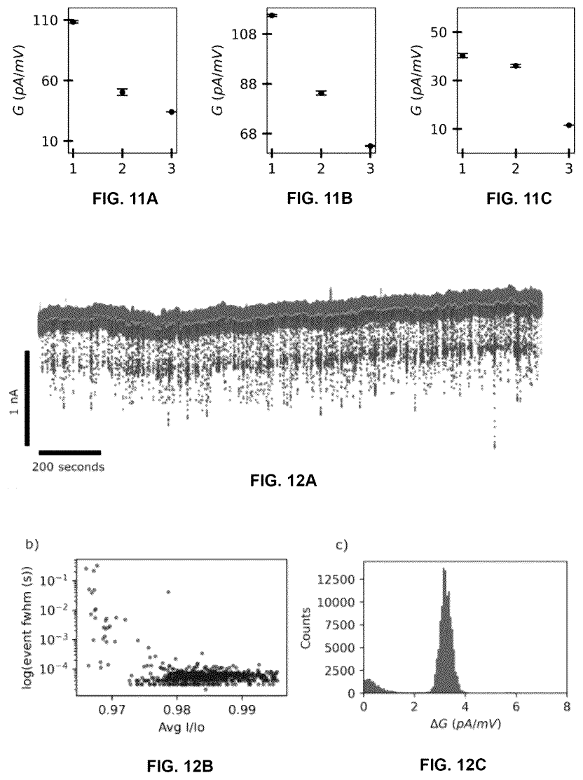

FIG. 12A, FIG. 12B, and FIG. 12C show measurements relating to DNA translocation through a pore functionalized with diol only (as shown in FIG. 3). In particular, these figures show translocation of 5 kbp NoLimits dsDNA which was tested to confirm that the modification of the pore with 2,2-Di(2-propyn-1-yl)-1,3-propanediol (DIOL) did not interrupt its ability to translocate analyte molecules. The results were gathered with an applied voltage of ˜50 mV (which is lower than the standard 200 mV that is commonly seen in the literature). FIG. 12A is an example current trace in blue with events highlighted in red. FIG. 12B shows event data for DNA revealing the relation between the blockage magnitude and dwell time of each event. FIG. 12C provides a histogram of the conductance of all event data points. The sample measured was 5 kbp NoLimits dsDNA in the conditions of 2 M KCl, 10 mM HEPES pH 7 electrolyte, with an applied voltage of negative 150 mV at 100 kHZ acquisition rate, 10k HZ Bessel filter and a gain of alpha:x2 beta:x1.

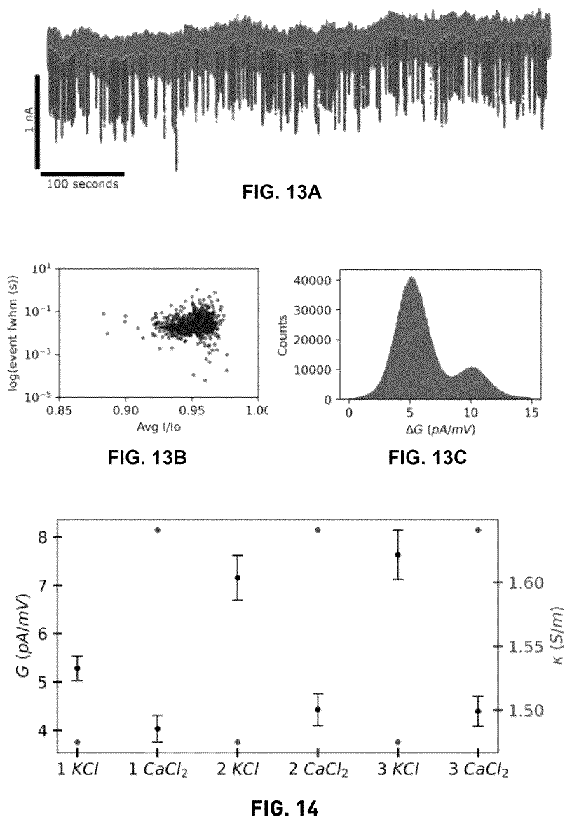

Next, FIG. 13A, FIG. 13B, and FIG. 13C show measurements of DNA translocation through pore after enhanced functionalization of the exemplary diol-functionalized pore with Adipoyl Chloride according to FIG. 10. FIG. 13A provides an example of a current trace in blue with events highlighted in red. The sample measured was 5 kbp NoLimits dsDNA in the conditions of 2 M KCl, 10 mM HEPES pH 7 electrolyte, with an applied voltage of negative 50 mV at 100 kHZ acquisition rate, 10k HZ Bessel filter and a gain of 2. FIG. 13B shows event data for DNA showing the relation between the blockage magnitude and dwell time of each event. FIG. 13C is a histogram of the conductance of all event data points.

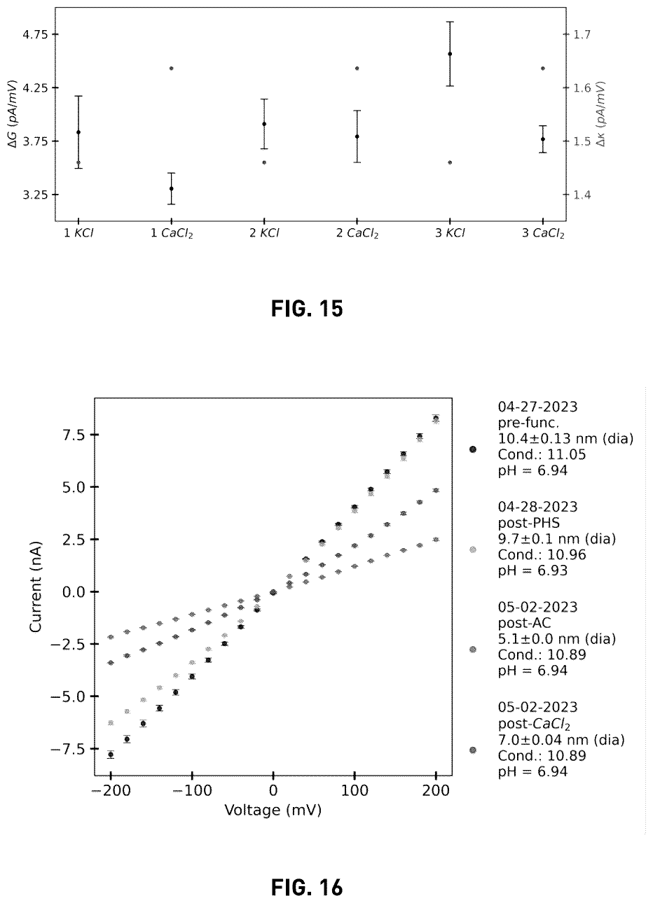

Calcium addition experiments, designed to confirm the presence of carboxylic acid groups, were done in 0.1 M KCl with a calcium addition concentration of 0.010 M at pH 8 and results are shown in FIGS. 14 and 15. The 2,2-Di(2-propyn-1-yl)-1,3-propanediol (DIOL) molecule was synthesized from malonic acid through alkylation and reduced to the alcohol form, FIG. 2. Acyl chloride functionalization was achieved by submerging the pore in neat adipoyl chloride for 1-24 hours (see FIG. 9).

Because a key part of the reconfiguration process (in the exemplary embodiment) is the carboxylic acid form, verifying the presence and ability to restore the carboxylic acid form was essential. FIG. 15 shows the results of an experiment to check the presence of a carboxylic acid termination. Addition of calcium chloride to the carboxylic acid terminated pore surface was achieved by swapping between a 0.1 M KCl solution and a 0.1 M KCl, 0.01 M CaCl₂ solution at pH 5.5. Specifically, the adipoyl chloride coated pore was exposed to 0.1 M potassium chloride electrolyte and the conductance was measured. The pore was then exposed to an electrolyte solution containing 0.1 M potassium chloride and 0.01 calcium chloride and the conductance was measured. The pore was exposed to each solution three times with the conductance measured each time. The trend seen in the nanopore's conductance was the opposite of the bulk solution conductivities. The addition of calcium increased the bulk conductivity of the electrolyte solution but reduced the current measured through the pore. Pore conductance is shown in black and bulk electrolyte conductivity is shown in red. The conductance of the pore dropped each time the calcium was introduced. Conductance of the pore was calculated from I/V curves while the bulk electrolyte conductivity was measured with a conventional conductivity probe and meter.

Importantly, FIG. 15 provides conductance measurements that validated reversible formation of a calcium acetate at the carboxylic acid terminations of the functionalized pore, a key feature of the present disclosure, namely, the ability to recycle the system to the carboxylic acid state in preparation for binding a different Coupling partner 116.

Nanopore diameters were calculated using their conductance, G, obtained from I/V measurements in 1 M KCl, 10 mM HEPES solutions at pH 7 and calculated with the following Equation:

$$d = ( \frac{G}{2 \cdot K} ) ( 1 + \sqrt{( 1 + \frac{16 \cdot K \cdot L}{π \cdot G} )} ) .$$

FIG. 16 illustrates Nanopore AC I/V curves for a typical SiNₓ nanopore in 1 M KC;, 10 mM HEPES, at pH˜7 (specific pH noted in the figure). The “pre-func” pore means the as-formed controlled dielectric breakdown pore, “post-PHS” or “post-photohydrosilyation” means after coating with 2,2-di(2-propyn-1-yl)-1,3-propanediol by photohydrosilylation, “post-AC” is after treating with adipoyl chloride, and “post-CaCl₂” means after exposing to calcium chloride salt. A larger slope of the I/V curve indicates a larger conductance: the as-formed pore diameter is reduced by coating with the diol. The conductance is further reduced after exposure to the adipoyl chloride because of the increased film thickness. The terminal group hydrolyzes to a carboxylic acid which can then complex with divalent calcium ions during treatment by solutions of calcium chloride.

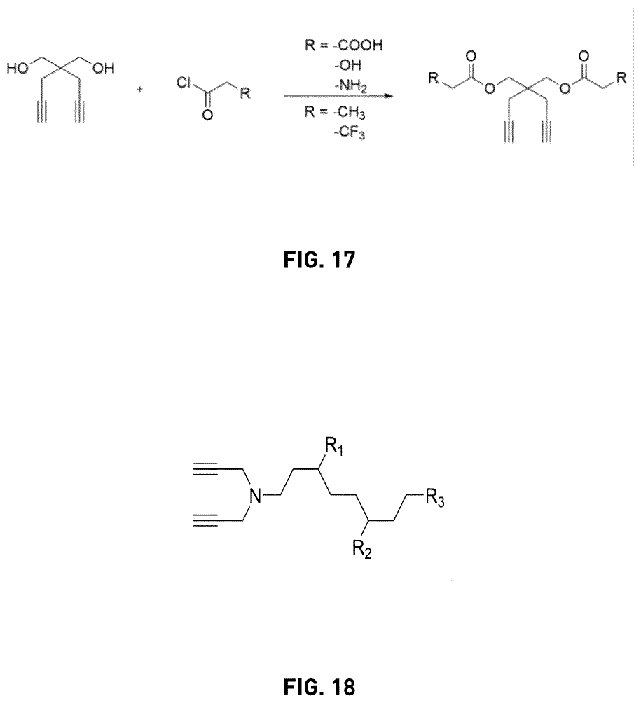

Significant variations in secondary functionalization with the custom DIOL example can be achieved by using acyl halides other than adipoyl chloride as shown in FIG. 17. Some examples can provide direct pathways to carboxylic acids, primary amines, alkanes or halogenated groups, as is known in the art.

Moreover, a variety of compounds, markedly different from the custom DIOL are capable of photohydrosilylation. For example, the amine compound of FIG. 18 provides a coating whose molecules have a high level of conformational flexibility.

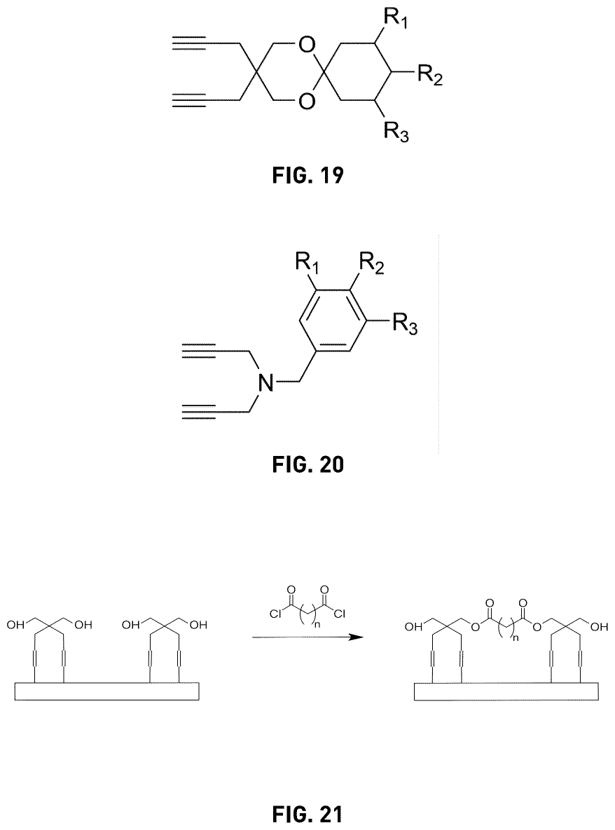

In contrast, the exemplary cyclic ether based stabilizing compound of FIG. 19 has a rigid structure having no net charges, providing corresponding surface changes.

On the other hand, a molecule such as the one shown in FIG. 20 offers rigidity through the planar benzene ring and provides the capability of aromatic interaction with analytes, with other molecules in the coating, and with R-substituents.

Creating a surface containing a significant population of coupling partner molecules not only attached to the stabilizing compound, but also crosslinked with stabilizing molecules will enhance stability and robustness of a nanopore, according to a further feature of the present invention. FIG. 21 shows that adipoyl chloride treatment as a coupling partner applied to the exemplary custom DIOL stabilizing compound is able to produce significant crosslinking. The ability of the coupling partner to crosslink can be modulated by selecting appropriate terminating groups (i.e., the R1, R2, an R3 components of the exemplary molecules in FIG. 18, FIG. 19, or FIG. 20) so as to place the appropriate binding sites of the coupling partners in close proximity after coupling to the stabilizing compound-or linkers of various lengths. Many variations of chemistry and geometry can be achieved through this scheme.

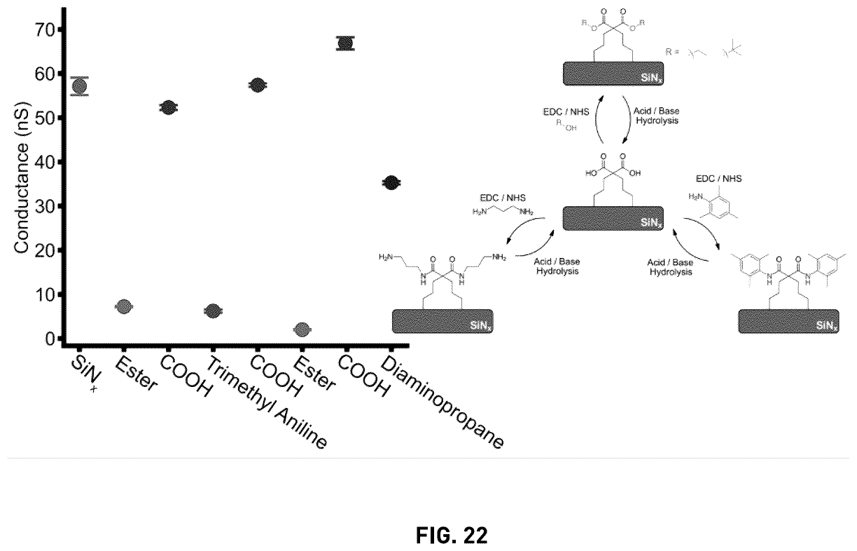

FIG. 22 illustrates pore conductance charted at various steps of fabrication of silicon nitride based nanopores according to one exemplary reversible functionalization scheme of the present disclosure. The reactants used for this illustration are among many possible sets, so this example is not meant to be limiting. First, conductance in units of nanoSiemens (nS) was plotted for a freshly fabricated nanopore as shown. Next, the highly reduced conductance of the ester configuration achieved according to the reactions scheme of FIG. 7 is shown. The conductance rises again in response to the hydrolysis of the ester to the carboxylic acid terminated form. The carboxylic acid form is the principal form for the reversible functionalization scheme. The carboxylic acid form, functioning as an adhesion layer, supports reactions with numerous varieties of coupling partners which can be bound and then removed to restore the carboxylic acid form. In the diagram, the next plot point shows conductance similar to that of the ester form after coupling trimethyl aniline. The next step shows a return via hydrolysis to the carboxylic acid form, with a corresponding increase in conductivity. The subsequent step demonstrates a return to the ester state, followed by functionalization with diaminopropane. Heterogeneous mixtures of coupling partners can be employed to tune the results. Overall, the figure illustrates a versatile approach to stabilizing and functionalizing nanopore, enhanced by providing additional reversible functionalization using a coupling partner.

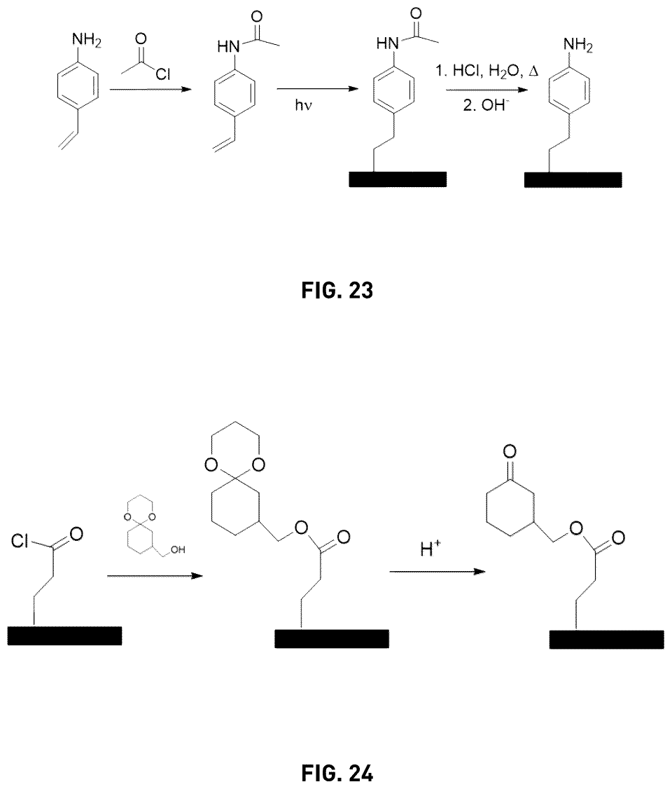

FIG. 23 provides an example of how to provide an alternative approach to customizing pore functionality—namely, combining secondary functionalization with the ability to modify the coupling partner directly rather than replacing the coupling partner as in FIG. 22. In this illustrative example, a cyclic ether is originally used as a coupling partner. Rather than using conditions which would remove the cyclic ether coupling partner from the adhesion layer by breaking the ester bond, acidic conditions that open the ether ring to form a ketone can be used to modify the original coupling partner molecule. Thus, the secondary functionalization layer is modified in situ.

FIG. 24 provides an illustrative example of how to create a reactive coupling site that otherwise could not be used on an adhesion layer. In the example, an amino group is desired as a coupling site on the adhesion layer. However, under the conditions needed for photohydrosilylation to bond the molecule to the SiNₓ, there would have been significant bonds between the nitrogen and the silicon formed creating a heterogeneous adhesion layer not desired in the example. To avoid that reaction, the amino group is first protected with a molecule possessing an adipoyl moiety. That “protecting group” prevents that amine group from reacting with a substrate when the molecule is bound to the SiNₓ through photohydrosilylation. Once the protected molecule has been attached to the surface, the protecting group is chemically removed (in this case, under aqueous conditions), allowing the much more reactive amino group to become available as a coupling site for a chosen secondary functionalization molecule.

The present disclosure and exemplary embodiments demonstrate a way to create highly controlled, reversibly functionalized, and stabilized nanopores. In an exemplary embodiment, a custom molecule tailored to the SiNₓ nanopore was synthesized and attached with the photohydrosilylation reaction. The design of the custom molecule produced a molecule having very high surface attachment, stability, functionality, and the ability to couple to additional chemicals. Surface attachment of a stabilizing compound may also provide an effective adhesion layer to counter the known instability of SiNₓ-based nanopores. The stabilizing compound also adds base functionality enabling these modified nanopores to be useful tools for polymer (or biopolymer) analysis and other selective nanopore applications. This adhesion layer also provides a base for subsequent enhanced functionalization through coupling partners. The approach disclosed for the stabilizing layer's coupling site provides a user-friendly chemical platform for enhanced functionalization using well-known chemistry to bind coupling partners. While the examples chosen here work well with conditions suitable for biologically relevant analytes, the principles of the invention can be readily extended to more industrially focused systems.

The invention, thus conceived, is susceptible of numerous modifications and

variations, all of which are within the scope of the appended claims. Moreover, all the details may be substituted by other, technically equivalent elements.

In practice the materials employed, provided they are compatible with the specific use, and the contingent dimensions and shapes, may be any according to requirements and to the state of the art.

Where technical features mentioned in any claim are followed by reference signs, such reference signs have been inserted for the sole purpose of increasing the intelligibility of the claims and accordingly such reference signs do not have any limiting effect on the interpretation of each element identified by way of example by such reference signs.

All publications and patent literature described herein are incorporated by reference in entirety to the extent permitted by applicable laws and regulations

1. A nanostructure comprising:
(a) a silicon-nitride substrate comprising a first surface and a second surface separated by a distance between 0.5 and 100 nm defining a substrate thickness;
(b) one or more nanopores comprising a nanoscale opening through the substrate, of between about 0.5 nm and about 100 nm in diameter, defining an interior surface; and
(c) an adhesion layer, comprising a carboxylic acid derivative of 2,2-Di(2-propyn-1-yl)-1,3-propanediol having one or more reactive alkyne or alkene regions and one or more hydroxyl bonding sites as a stabilizing compound disposed at least over the interior surface, and covalently bound through a carbon-silicon bond to the substrate through said one or more reactive alkyne or alkene regions.

2. The nanostructure of claim 1, further comprising a functional enhancement layer disposed over the adhesion layer formed by binding a coupling partner selected from the group consisting of trimethyl aniline, diaminopropane, click chemistry, n-propanephosphonic acid anhydride, various bifunctional boronic acids, oxo-acids, vinylphosphonic acids, chiral cyclopentenol, nicotinic acids and derivatives, aliphatic amines, and various heterocycles, to the adhesion layer.

3. A substrate comprising:
(a) a first surface and a second surface separated by a distance between 0.5 and 100 nm defining a substrate thickness;
(b) one or more nanopores comprising a nanoscale opening through the substrate, of between about 0.5 nm and about 100 nm in diameter, defining an interior surface, and;
(c) an adhesion layer, disposed at least over the interior surface, comprising a stabilizing compound having at least one bonding site and at least one coupling site, wherein said stabilizing compound is covalently bound to the substrate through said at least one bonding site.

4. The substrate of claim 3, further comprising a functional enhancement layer bound to the adhesion layer, said functional enhancement layer comprising a coupling partner having a coupling bonding site, wherein said functional enhancement layer is bound to said adhesion layer through coupling between said coupling bonding site on said coupling partner and said at least one coupling site on said stabilizing compound.

5. The substrate of claim 3 wherein the stabilizing compound comprises 2,2-Di(2-propyn-1-yl)-1,3-propanediol.

6. The substrate of claim 3, comprising two or more chemically distinct stabilizing compounds forming a heterogeneous adhesion layer.

7. The substrate of claim 3 wherein at least one stabilizing compound has a carboxylic acid termination.

8. The substrate of claim 3 wherein at least one stabilizing compound has two or more bonding sites.

9. The substrate of claim 8, wherein, in the at least one stabilizing compound, at least one of the two or more bonding sites is bound to another molecule of the adhesion layer rather than the substrate, resulting in cross-linking within the adhesion layer.

10. The substrate of claim 3 wherein the adhesion layer surface is rich with sites capable of reversible hydrolysis and esterification with one or more of the coupling partners.

11. The substrate of claim 3 wherein the substrate is silicon nitride.

12. The substrate of claim 3 wherein the nanopore has an average diameter between about 0.5 nm and about 100 nm.

13. The substrate of claim 3 wherein each molecule of the one or more of the stabilizing compounds comprises more than one exposed functional group.

14. The substrate of claim 3 wherein one of the one or more bonding sites is bonded to a molecule of one of the coupling partners.

15. The substrate of claim 3 wherein the stabilizing compounds are bonded to the substrate through a photohydrosilyation reaction.

16. The substrate of claim 3 wherein the stabilizing compounds' bonding site is proximate to an unsaturated C—C bond.

17. The substrate of claim 3 wherein the adhesion layer alters the effective hydrophobicity of the one or more nanopores.

18. The substrate of claim 3 wherein the coupling partner alters the effective conductivity of the one or more nanopores.

19. The substrate of claim 3 wherein one or more of the coupling partners is either trimethyl aniline or diaminopropane.

20. The substrate of claim 4 wherein at least one coupling partner is bound to the adhesion layer through either an ester bond or an amide bond.

21. The substrate of claim 4 wherein one or more of the coupling partners is an acyl chloride.

22. The substrate of claim 3 wherein one or more of the coupling partners is a compound capable of selective bonding to an antigen.

23. The substrate of claim 1 wherein one or more of the stabilizing compounds comprise aromatic groups to stabilize the coating.

24. The substrate of claim 1 wherein the nanostructure is shelf-stable for at least one week.

25. (canceled)

26. (canceled)

27. (canceled)

28. (canceled)

29. (canceled)

30. (canceled)
